(19) United States
(12) Patent Application Publication
(10) Pub. No.: US 2025/0264707 A1
(43) Pub. Date: Aug. 21, 2025

(54) OBSERVATION APPARATUS

(71) Applicant: NIKON CORPORATION, Tokyo (JP)

(72) Inventors: Hitoshi KAWAI, Saitama-shi (JP); Yamato NIITANI, Yokohama-shi (JP); Yosuke FUJIKAKE, Yokohama-shi (JP)

(73) Assignee: NIKON CORPORATION, Tokyo (JP)

(21) Appl. No.: 19/202,126

(22) Filed: May 8, 2025

Related U.S. Application Data
(63) Continuation of application No. PCT/JP2023/042430, filed on Nov. 27, 2023.

(30) Foreign Application Priority Data
Dec. 20, 2022 (JP) 2022-203038

(51) Int. Cl. G02B 21/06

(52) U.S. Cl. CPC G02B 21/06

(57) ABSTRACT

There is provided an observation apparatus which observes a microfluidic device having an observation target region that is a target of observation, and a structure, the observation apparatus including: an observation optical system, in which the observation optical system has a mask which blocks at least a part of light that is subjected to an optical effect by the structure that exists partway on a path of a light beam reaching the observation optical system, in light beams from the observation target region, each of which is the light beam.

The contents of the following patent application(s) are incorporated herein by reference:
NO. 2022-203038 filed in JP on Dec. 20, 2022
NO. PCT/JP2023/042430 filed in WO on Nov. 27, 2023.

### BACKGROUND

### 1. Technical Field

The present invention relates to an observation apparatus.

### 2. Related Art

Patent Document 1 discloses a microscope which is capable of using an ordinary objective lens for special observation, and which can enhance degree of freedom in replacement of the objective lens. Patent Document 2 discloses a microfluidic device observation apparatus and a microfluidic device observation method for observing a specimen present in one or more flow paths, the flow paths being arranged in a microfluidic device. Prior Art Documents

### Patent Document

Patent Document 1: Japanese Patent Application Publication No. 2009-115902
Patent Document 2: International Publication No. WO 2020/021604

### GENERAL DISCLOSURE

In a first aspect of the present invention, there is provided an observation apparatus which observes a microfluidic device having an observation target region that is a target of observation, and a structure, the observation apparatus including: an observation optical system, in which the observation optical system has a mask which blocks at least a part of light that is subjected to an optical effect by the structure that exists partway on a path of a light beam reaching the observation optical system, in light beams from the observation target region, each of which is the light beam.

The observation optical system may have an objective lens which collects a light beam from the observation target region, and the mask may be arranged at a position corresponding to a rear focal position of the objective lens.

The observation apparatus may further have an illumination optical system which illuminates the observation target region.

The observation optical system may have a plurality of masks in which regions that are blocked are different from each other, and the plurality of masks may be configured to be switchable.

The observation apparatus may further have a control device, in which the control device may automatically switch the plurality of mask according to a position of the observation target region or a type of the microfluidic device that is observed.

The mask may be configured to be movable in a direction along an optical axis of the observation optical system.

The observation apparatus may further have a spatial light modulator, and the mask may be formed by the spatial light modulator.

The mask may be selected based on information received by the observation apparatus.

It should be noted that the summary clause does not necessarily describe all necessary features of the embodiments of the present invention. In addition, the invention may also be a sub-combination of the features described above.

### BRIEF DESCRIPTION OF THE DRAWINGS

FIG. 1 is a top view showing an example of a schematic configuration of a microfluidic device 100 in the present embodiment.

FIG. 2 is a side view showing an example of the schematic configuration of the microfluidic device 100 in the present embodiment.

FIG. 3 shows an example of a schematic configuration of an observation apparatus 300 according to the present embodiment.

FIG. 4 is a top view showing an example of a schematic configuration of a mask unit 315 in the present embodiment.

FIG. 5 is a schematic diagram showing a first mask 351 in the present embodiment.

FIG. 6 is a schematic diagram showing a second mask 352 in the present embodiment.

FIG. 7 is a schematic diagram showing a third mask 353 in the present embodiment.

FIG. 8 is a schematic diagram showing a fourth mask 354 in the present embodiment.

FIG. 9 is a flowchart showing an example of an operation of the observation apparatus 300 in the present embodiment.

FIG. 10 shows an example of a computer 2200.

### DESCRIPTION OF EXEMPLARY EMBODIMENTS

Hereinafter, the present invention will be described through embodiments of the invention. The embodiments described below are not intended to limit the invention within the scope of the claims. Not all combinations of features described in the embodiments are essential for the solution of the invention.

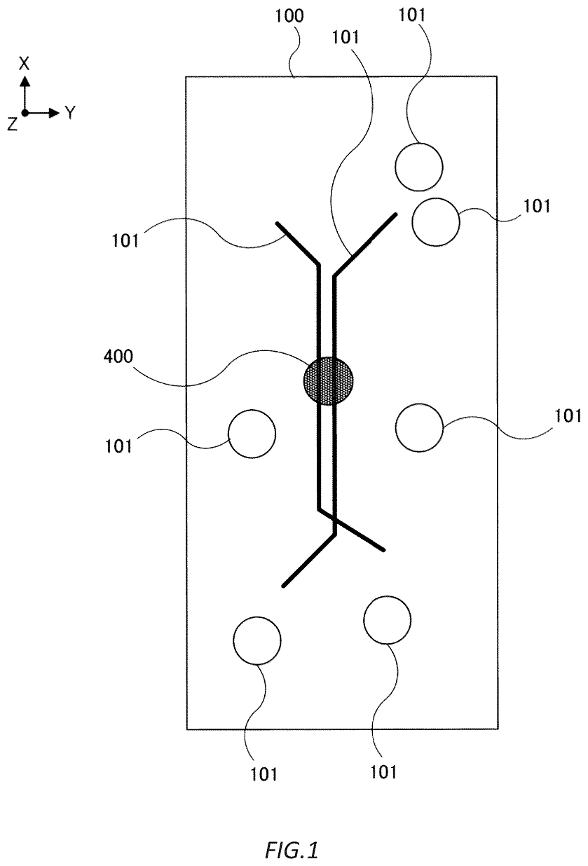

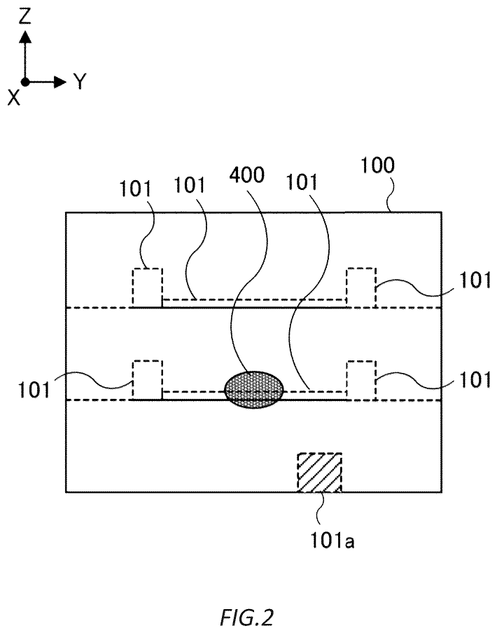

FIG. 1 is a top view showing an example of a schematic configuration of a microfluidic device 100 in the present embodiment. FIG. 2 is a side view showing an example of the schematic configuration of the microfluidic device 100 in the present embodiment. In the figure, an XYZ coordinate system is shown below. As shown in FIG. 1 and FIG. 2, the microfluidic device (microfluidic devices) 100 has a substantially rectangular parallelepiped shape. It should be noted that the present invention is also applicable to a microfluidic device having another three-dimensional shape.

The microfluidic device 100 refers to a chip for: arranging, on a small substrate, a sample corresponding to a biological microscopic substance such as DNA, a protein, a cell, a cell cluster (a spheroid, an organoid, or the like), or a tissue; and analyzing a gene defect, a protein distribution, a reaction pattern, or the like. The microfluidic device 100 in the present embodiment is also called an organ chip (an organ on a chip), a biological function chip (an organ on a chip), micro physiological systems (MPS), a bio chip, a microfluidic chip, a micro chip, a cell culture chip, a micro flow path chip, or the like. The microfluidic device 100 is an object of observation.

The microfluidic device 100 is used, as an example, for culturing and analyzing a cell, a cell cluster, or a tissue. Further, a chemical substance (a drug) is added to be used for an evaluation, an analysis, or the like of a reaction with a cultured cell. The microfluidic device 100 may include both of: a portion in which an organ cell is cultured to express a biological function; and a device main body in which the organ cell is not cultured and which is “empty”.

The microfluidic device 100 can be manufactured, for example, by using a stereolithography three-dimensional printing technique and a solution casting molding process. In addition to the techniques mentioned above, the microfluidic device 100 can be manufactured by another micro processing technique, such as Micro Electro Mechanical Systems (MEMS), for example.

As shown in FIG. 1 and FIG. 2, the microfluidic device 100 has a plurality of layers, and in each layer of the microfluidic device 100, a plurality of structures 101 such as micro flow paths are arranged. For example, when a small intestine model is constructed, in a micro flow path, an upper channel, mucus, a suction channel, a small intestine epithelial cell, a porous membrane, an endothelial cell, and a lower channel are stacked in order, to construct the small intestine model. It should be noted that the microfluidic device 100 is not limited to a case of having a plurality of layers.

Each layer of the microfluidic device 100 is, as an example, constituted by a substrate. The substrate may be constituted, for example, by glass. The substrate may be constituted, for example, by a resin material such as polymethyl methacrylate (PMMA), polycarbonate (PC), cycloolefin copolymer (COC), cycloolefin polymer (COP), polystyrene (PS), or silicon. The microfluidic device 100 may be hollow, or may be solid. The microfluidic device 100 may have a cover which covers the entire microfluidic device 100. It is desirable that the microfluidic device 100 is transparent for illumination light and observation light.

The microfluidic device 100 has an observation target region 400 that is a target of observation. The observation target region 400 of the microfluidic device 100 is a region that a user desires to observe in the microfluidic device 100, and for example, is a region in which a cultured cell is arranged. In the observation target region 400, for example, in addition to the cultured cell, a structure 101 such as a micro flow path (for example, a porous membrane or the like) may be arranged. The observation target region 400 may be a part of a region of the porous membrane of the microfluidic device 100. An arrangement (or a position) of the observation target region 400 in the microfluidic device 100 varies for each individual microfluidic device 100, and is able to be grasped in advance.

The microfluidic device 100 has an obstacle that obstructs the observation of the observation target region 400. The obstacle is, for example, the structure 101 such as the micro flow path disposed on each layer of the microfluidic device 100. When the observation target region 400 is observed, by the obstacle existing between the observation target region 400 and an objective lens, the observation light from the observation target region 400 is partially blocked or scattered by the obstacle, which obstructs the observation.

It should be noted that not all the structures 101 which are disposed in the microfluidic device 100 become obstacles, and whether or not the structure 101 is an obstacle is determined by a positional relationship between the observation target region 400 and the structure 101, in particular by an optical positional relationship in consideration of a field of view and an aperture of the objective lens, as well. In FIG. 2, as an example, a structure 101a which is hatched is set to be the obstacle. Then, another structure 101 is not set to be the obstacle. That is, the obstacle is a structure that exists partway on a path of the observation light reaching the objective lens from the observation target region, and causes absorption, reflection, and/or scattering (optical effects).

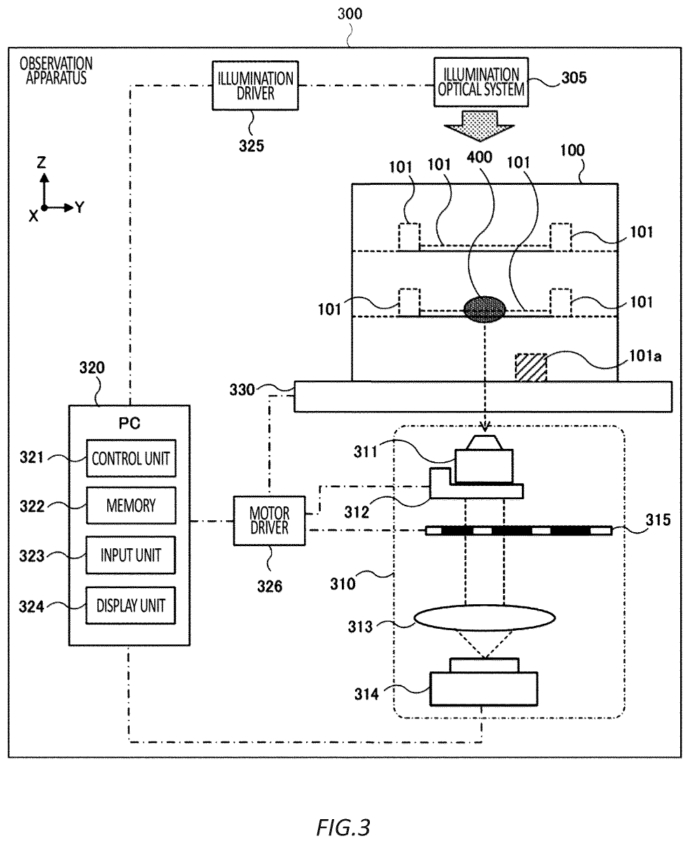

FIG. 3 shows an example of a schematic configuration of an observation apparatus 300 according to the present embodiment. The observation apparatus 300 in the present embodiment may be an inverted microscope, as an example. As shown in FIG. 3, the observation apparatus 300 includes an illumination optical system 305, an observation optical system 310, a PC 320, a first stage 330, a motor driver 326, and an illumination driver 325. The microfluidic device 100 is placed on a first stage 330 of the observation apparatus 300, and the microfluidic device 100 is movable in an X direction and a Y direction by moving the first stage 330 in the X direction and the Y direction. The first stage 330 is provided with a hole for observation for transmitting the observation light from the observation target region 400. It should be noted that for simplicity, a main optical axis is shown and the details of the observation light are omitted.

The observation optical system 310 has an objective lens 311, a second stage 312 on which the objective lens 311 is placed, an imaging lens 313, a two-dimensional detector 314, and a mask unit 315. The second stage 312 is movable in a Z direction (a height direction), and a position of the objective lens 311 is adjustable in the Z direction. The two-dimensional detector 314 detects the observation light from the observation target region 400. The two-dimensional detector 314 is, as an example, an image sensor such as a Charge Coupled Device (CCD) image sensor or a scientific Complementary Metal Oxide Semiconductor (sCMOS) image sensor. It should be noted that the observation optical system 310 may further include optical members such as a condenser lens and a dichroic mirror, in addition to the objective lens 311.

The mask unit 315 is arranged at a position corresponding to a rear focal position of the objective lens 311. The position corresponding to the rear focal position may be the rear focal position itself, or may be a position optically conjugate with the rear focal position. For the rear focal position of the objective lens 311, with the rear focal position which is theoretically uniquely determined, being set as the center, a shift up to 100 times a focal depth of the objective lens 311 in an optical axis direction, is in a permissible range. It should be noted that in other words, the rear focal position is an exit pupil position.

The PC 320 has a control unit 321 having a CPU and a memory 322, and the control unit 321 reads and executes a control program stored in the memory 322, thereby controlling an operation of the observation apparatus 300. The PC 320 has: an input unit 323 which receives various types of instructions and settings and the like from the user, and transmits them to the control unit 321 of the PC 320; and a display unit 324 which receives a command from the control unit 321, and displays various types of dialogues and the like to the user.

As shown by a dash-single dotted line in FIG. 3, the PC 320 is connected to the first stage 330, the second stage 312, and the mask unit 315 in the observation apparatus 300, via the motor driver 326; and is able to control operations of each stage and the mask unit 315 by controlling the motor driver 326. In addition, as indicated by the dash-single dotted line in FIG. 3, the PC 320 is connected to the two-dimensional detector 314, and receives a detected image.

The PC 320 can control one or more of an illumination intensity, an illumination position, and an illumination timing of the illumination optical system 305. As shown by the dash-single dotted line in FIG. 3, the PC 320 is connected to the illumination optical system 305 via the illumination driver 325; and is able to control turning on and turning off of an illumination member, the illumination intensity, or the like in the illumination optical system 305, by controlling the illumination driver 325. In addition, the PC 320 is capable of controlling such as controlling of turning on and turning off the illumination member in the illumination optical system 305 at a predetermined timing. Radiation light radiated to the observation target region 400 via the illumination optical system 305 is, for example, excitation light from an ultraviolet range to an infrared range in a case of fluorescence observation; and is illumination light in a visible range to the infrared range in a case of bright-field observation and dark-field observation.

It should be noted that the observation light that is detected by the two-dimensional detector 314 is fluorescence generated by a fluorescent dye in the observation target region 400 being excited by the illumination light emitted from the illumination optical system 305, in a case of the fluorescence observation; is light obtained by the illumination light emitted from the illumination optical system 305 being transmitted, diffracted, and scattered through the observation target region 400, in a case of the bright-field observation; and is light obtained by the illumination light emitted from the illumination optical system 305 being diffracted and scattered in the observation target region 400, in a case of the dark-field observation. Whether to perform the bright-field observation or the dark-field observation can be set by: an optical characteristic of the observation optical system 310 such as a NA of the objective lens 311; and a direction of illumination light from the illumination optical system 305 to the observation target region 400; and others.

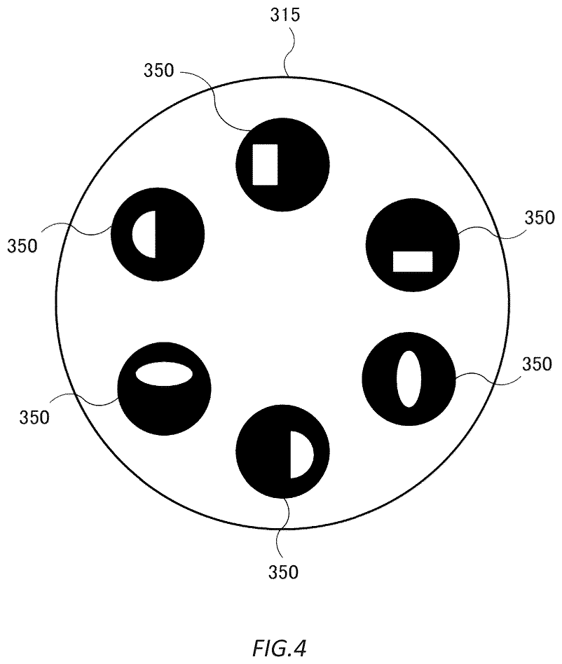

FIG. 4 is a top view showing an example of a schematic configuration of a mask unit 315 in the present embodiment. The mask unit 315 is a unit in which a plurality of masks 350 are arranged on a rotating holder. The plurality of masks 350 block at least a part of light that is absorbed, reflected, and/or scattered (subjected to optical effects) by the obstacle, in light beams of the observation light from the observation target region 400. Each of the plurality of masks 350 has a region which blocks the light beam from the observation target region 400, and a region through which the light beam is transmitted; and the regions have shapes different from each other. As examples, FIG. 4 shows: the mask 350 which has a transmissive region (shown as a white space in the figure) of an elliptical shape; the mask 350 which has a transmissive region of a semicircular shape; and the mask 350 which has a transmissive region of a rectangular shape. It should be noted that the mask unit 315 may be configured to be movable in the optical axis direction.

The PC 320 automatically switches between the plurality of masks 350 according to a type of the microfluidic device 100 that is observed. That is, the PC 320 rotates the rotating holder via the motor driver 326 to select an appropriate type of mask 350 in the observation, and arranges it at a position corresponding to the rear focal position of the objective lens 311. It should be noted that the mask 350 may adopt a shape other than that shown in FIG. 4. In addition, when there are two or more observation target regions 400 in the microfluidic device 100, the mask 350 may be selected based on the observation target region 400 of the microfluidic device 100 input to the observation apparatus 300. In addition, the mask 350 may be switched manually. Further, when the objective lens 311 is switched (magnification is changed), the PC 320 may move the rotating holder in the optical axis direction via the motor driver 326.

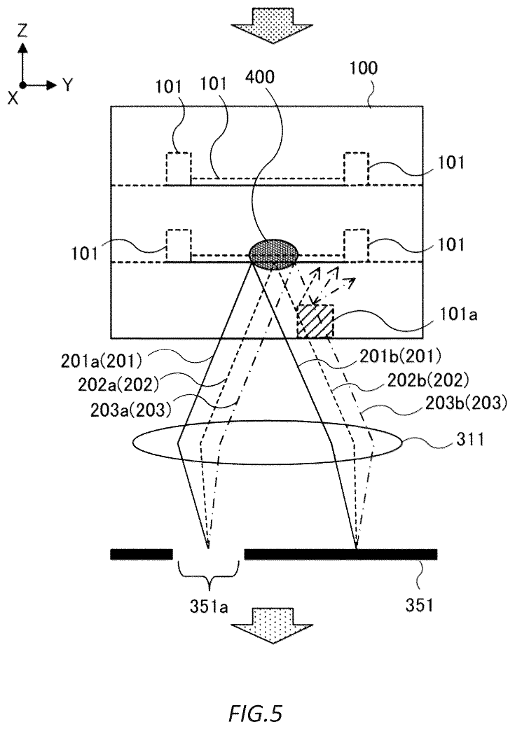

FIG. 5 is a schematic diagram showing a first mask 351 in the present embodiment. FIG. 5 shows the microfluidic device 100, the observation light from the observation target region 400 of the microfluidic device 100, the objective lens 311, and the first mask 351. In FIG. 5, another component in the observation apparatus 300 is omitted.

By the illumination light being radiated from the illumination optical system 305 to the observation target region 400 of the microfluidic device 100, the observation light is emitted from the observation target region 400 as shown in FIG. 5. In FIG. 5, the observation light that is emitted from a left end of the observation target region 400 is shown as observation light 201; the observation light that is emitted from the center of the observation target region 400 is shown as observation light 202; and the observation light that is emitted from a right end of the observation target region 400 is shown as observation light 203. In addition, the observation light 201, 202, 203 are divided into light that is emitted in a lower left direction of the observation target region 400; and light that is emitted in a lower right direction of the observation target region 400.

In FIG. 5, a left end and a right end of the observation light 201, which is emitted from the left end of the observation target region 400 and is incident on the objective lens 311, are respectively set as observation light 201a and observation light 201b. In addition, a left end and a right end of the observation light 202, which is emitted from the center of the observation target region 400 and is incident on the objective lens 311, are respectively set as observation light 202a and as observation light 202b. Further, a left end and a right end of the observation light 203, which is emitted from the right end of the observation target region 400 and is incident on the objective lens 311, are respectively set as observation light 203a and observation light 203b.

In FIG. 5, the structure 101a which is an obstacle is arranged at a lower right position of the observation target region 400. Accordingly, a part (the observation light 202b, 203b) of the observation light 202, 203 which is emitted in the lower right direction of the observation target region 400, is reflected, scattered, or the like by the structure 101a, and a sufficient amount of light does not reach the two-dimensional detector 314. If the observation light is detected in such a state by the two-dimensional detector 314, and is turned into an image, the image has unevenness and brightness varies from place to place.

Accordingly, in the present example, the first mask 351 is arranged to remove the observation light that is reflected, scattered, or the like by the structure 101a which is an obstacle, and to generate an image having approximately the same overall brightness and little unevenness. The first mask 351 is an example of the mask 350 in FIG. 4. As shown in FIG. 5, the first mask 351 is provided with a transmissive region 351a on a left side; and has a shape in which the observation light that is emitted in the lower left direction of the observation target region 400, is transmitted, and the observation light that is emitted in the lower right direction of the observation target region 400, is blocked. This makes it possible to detect the observation light obtained by removing, as much as possible, the observation light that has been subjected to the optical effects (influences) of the structure 101a which is an obstacle, and to generate an image, and the image has approximately the same overall brightness and little unevenness.

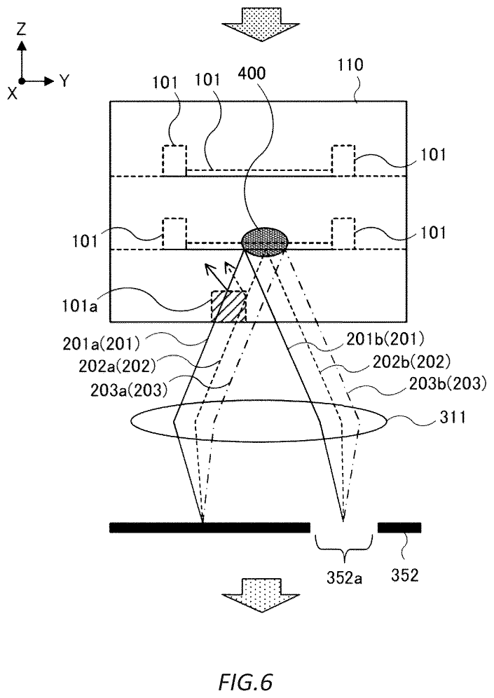

FIG. 6 is a schematic diagram showing a second mask 352 in the present embodiment. FIG. 6 shows a microfluidic device 110, a plurality of beams of observation lights from the observation target region 400 of the microfluidic device 110, the objective lens 311, and the second mask 352. In FIG. 6, another component in the observation apparatus 300 is omitted.

In FIG. 6, the structure 101a which is an obstacle is arranged at a lower left position of the observation target region 400. Accordingly, a part (the observation light 201a, 202a) of the observation light 201, 202 which is emitted in the lower left direction of the observation target region 400, is reflected, scattered, or the like by the structure 101a. In the present example, the second mask 352 is arranged. The second mask 352 is an example of the mask 350. As shown in FIG. 6, the second mask 352 is provided with a transmissive region 352a on a right side; and has a shape in which the observation light that is emitted in the lower right direction of the observation target region 400, is transmitted, and the observation light that is emitted in the lower left direction of the observation target region 400, is blocked.

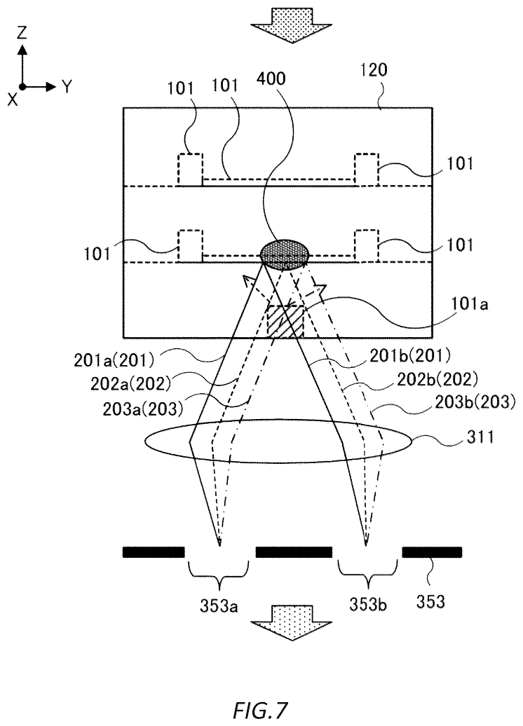

FIG. 7 is a schematic diagram showing a third mask 353 in the present embodiment. FIG. 7 shows a microfluidic device 120, a plurality of beams of observation lights from the observation target region 400 of the microfluidic device 120, the objective lens 311, and the third mask 353. In FIG. 7, another component in the observation apparatus 300 is omitted.

In FIG. 7, the structure 101a which is an obstacle is arranged at a position directly below the observation target region 400. Accordingly, a part (the observation light 202a, 203a, 201b, 202b) of the observation light 201, 202, 203 which is emitted in a downward direction directly below the observation target region 400, is reflected, scattered, or the like by the structure 101a. In the present example, the third mask 353 is arranged. The third mask 353 is an example of the mask 350. As shown in FIG. 7, the third mask 353 is provided with a transmissive region 353a and 353b on a left side and a right side, respectively; and has a shape in which the observation light that is emitted in the lower right direction and the lower left direction of the observation target region 400, is transmitted, and the observation light that is emitted in the downward direction directly below the observation target region 400, is blocked.

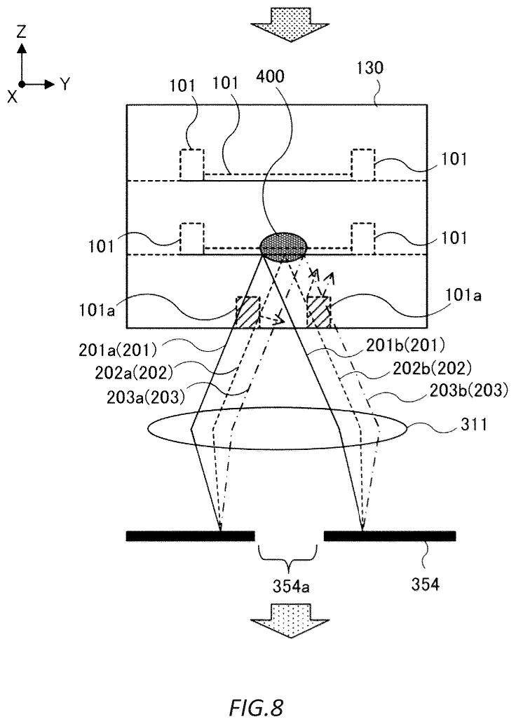

FIG. 8 is a schematic diagram showing a fourth mask 354 in the present embodiment. FIG. 8 shows a microfluidic device 130, a plurality of beams of observation lights from the observation target region 400 of the microfluidic device 130, the objective lens 311, and the fourth mask 354. In FIG. 8, another component in the observation apparatus 300 is omitted.

In FIG. 8, two structures 101a which are obstacles are respectively arranged at a lower left position and a lower right position of the observation target region 400. Accordingly, a part (the observation light 201a, 202a, 202b, 203b) of the observation light 201, 202, 203 which is emitted in the lower left direction and the lower right direction of the observation target region 400, is reflected, scattered, or the like by the structure 101a. In the present example, the fourth mask 354 is arranged. The fourth mask 354 is an example of the mask 350. As shown in FIG. 8, the fourth mask 354 is provided with a transmissive region 354a in the center; and has a shape in which the observation light that is emitted in the downward direction directly below the observation target region 400, is transmitted, and the observation light that is emitted in the lower left direction and the lower right direction of the observation target region 400, is blocked.

FIG. 5 to FIG. 8 have described the first mask 351 to the fourth mask 354 as examples of the mask 350; however, the mask 350 may have another shape and another transmissive region. The shape of the mask 350 may be determined according to a type of the microfluidic device 100 or the like which is observed. For the microfluidic device 100 or the like which is observed, the shape of the mask 350 may be determined based on at least one of a size, a range, and a position of the observation target region 400 in the microfluidic device 100 or the like; a size, a range, and a position of the structure 101a which is an obstacle in the microfluidic device 100 or the like; or a relative positional relationship between the observation target region 400 and the structure 101a which is an obstacle. The shape of the mask 350 may be determined based on the overall shape of the microfluidic device 100 or the like, or a material property such as a refractive index. The shape of the mask 350 may be determined based on a property of the illumination light of the illumination optical system 305 in the observation apparatus 300.

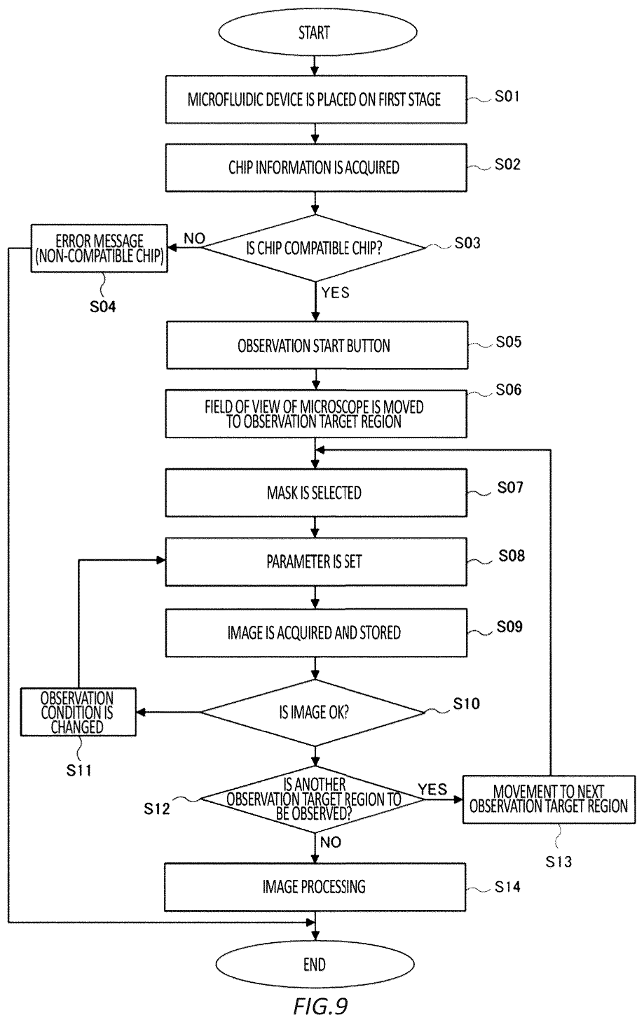

FIG. 9 is a flowchart showing an example of an operation of the observation apparatus 300 in the present embodiment. In step S01, the microfluidic device 100 is placed on the first stage 330. Subsequently, in step S02, chip information which is individual identification information of the microfluidic device 100, is acquired. The chip information is automatically acquired by the observation apparatus 300 reading identification information such as a barcode printed on a surface of the microfluidic device 100 or an embedded Radio Frequency Identification (RFID).

Subsequently, in step S03, it is determined whether the microfluidic device 100 is a compatible chip or a non-compatible chip based on the acquired chip information. The compatible chip is a chip for which the observation apparatus 300 retains the information on the microfluidic device 100, thereby retaining the information such as the arrangement and overall shape of the structure 101 inside the microfluidic device 100; and the non-compatible chip is a chip for which the observation apparatus 300 does not retain such information.

If the microfluidic device 100 is a non-compliant chip (NO in step S03), processing proceeds to next step S04, and an error message is displayed to the user to provide a notification that the microfluidic device 100 is the non-compliant chip. If the microfluidic device 100 is a compatible chip (YES in step S03), the processing proceeds to next step S05, and a display is performed to prompt the user to press an observation start button.

When the user presses the observation start button, in next step S06, a field of view of the microscope as the observation apparatus 300 is moved to the observation target region 400. The processing of step S06 is performed by the user manually adjusting the field of view of the microscope to the observation target region 400. However, the processing of step S06 may be performed automatically by the observation apparatus 300.

Subsequently, in step S07, the PC 320 selects the mask 350 which is suitable for the microfluidic device 100 that is an observation target. In a case of the compatible chip, the PC 320 stores, in advance, a table in which a chip ID and a mask ID that are assigned for each microfluidic device 100 (or each observation target region 400 of the microfluidic device 100), are associated with each other; and with reference to the table, the appropriate mask 350 is selected.

Subsequently, in step S08, the user performs another adjustment on the observation apparatus 300. Various types of parameters include the illumination intensity, the illumination position, and the illumination timing of the illumination optical system 305, a range of the field of view of the observation apparatus, and the like. Subsequently, in step S09, the PC 320 acquires and stores an image of the observation target region 400. Subsequently, in step S10, the user checks whether or not there is any problem with the acquired image. If there is a problem with the acquired image (NO in step S10), the processing proceeds to step S11; an observation condition is changed; the parameter is adjusted again in step S08; and an image of the observation target region 400 is acquired and stored again.

If there is no problem with the acquired image (YES in step S10), the processing proceeds to next step S12, and it is determined whether or not the next observation target region 400 exists in the microfluidic device 100. If there is the next observation target region 400 (YES in step S12), in next step S13, the field of view of the microscope is moved to the next observation target region, and the processing returns to step S07, and the processing of step S07 to the processing of step S10 are repeated. If there is no next observation target region (NO in step S12), the processing proceeds to next step S14, and the PC 320 performs a predetermined image processing on the stored image. At this time, predetermined analysis and evaluation processing such as counting the number of cells, calculating a cell density distribution, and determining whether the cell is alive or dead, may be performed. Once the image processing is performed on the stored image, the processing ends.

According to the observation apparatus 300 in the embodiment described above, in consideration of the observation target region 400 of the microfluidic device 100 which is observed, or an arrangement structure of the structure 101a which is an obstacle, the mask 350 which blocks at least a part of the observation light that is absorbed, reflected, and/or scattered (subjected to the optical effects) by the obstacle, in the observation light from the observation target region 400, is formed. This makes it possible to detect the observation light obtained by removing, as much as possible, the observation light that has been subjected to the optical effects (influences) of the structure 101a which is an obstacle, and to generate an image, and it is possible to generate the image having approximately the same overall brightness and little unevenness.

In the embodiment described above, the mask unit 315 is configured as a unit in which the plurality of masks 350 are arranged on the rotating holder. However, instead of the mask unit 315, a Spatial Light Modulator (SLM) of a liquid crystal type may be used to form a plurality of types of masks 350. The spatial light modulator is a device that modulates, for emitting, a spatial distribution of a property such as an amplitude, a phase, and a polarization of incident light, by electrically controlling each of a plurality of elements arranged two-dimensionally. By the PC 320 controlling a state of a liquid crystal for each pixel arranged two-dimensionally to select whether to transmit or block the incident light, it is possible for the spatial light modulator of the liquid crystal type to form the plurality of types of masks 350 having various transmissive regions.

By using the spatial light modulator of the liquid crystal type, it is possible to generate the mask 350 of any shape in real time. In addition, it is possible to correct the intensity and the phase of the observation light, and thus it is possible to function as a density filter, as well.

In addition, various embodiments of the present invention may be described with reference to flowcharts and block diagrams, wherein the block may serve as (1) a stage in a process in which an operation is performed, or (2) a section of an apparatus having a role of performing an operation. Certain stages and sections may be implemented by dedicated circuitry, programmable circuitry supplied together with computer-readable instructions stored on computer-readable media, and/or processors supplied together with computer-readable instructions stored on computer-readable media. The dedicated circuitry may include digital and/or analog hardware circuits, and may include integrated circuits (IC) and/or discrete circuits. The programmable circuitry may include a reconfigurable hardware circuit including logical AND, logical OR, logical XOR, logical NAND, logical NOR, and other logical operations, a memory element or the like such as a flip-flop, a register, a field programmable gate array (FPGA) and a programmable logic array (PLA), or the like.

A computer-readable medium may include any tangible device that can store instructions to be executed by a suitable device, and as a result, the computer-readable medium having instructions stored thereon includes a product including instructions that can be executed in order to create means for executing operations specified in the flowcharts or block diagrams. Examples of the computer-readable medium may include an electronic storage medium, a magnetic storage medium, an optical storage medium, an electromagnetic storage medium, a semiconductor storage medium, and the like. More specific examples of the computer-readable medium may include a floppy (registered trademark) disk, a diskette, a hard disk, a random access memory (RAM), a read-only memory (ROM), an erasable programmable read-only memory (EPROM or flash memory), an electrically erasable programmable read-only memory (EEPROM), a static random access memory (SRAM), a compact disc read-only memory (CD-ROM), a digital versatile disc (DVD), a Blu-ray (registered trademark) disc, a memory stick, an integrated circuit card, or the like.

The computer-readable instruction may include: an assembler instruction, an instruction-set-architecture (ISA) instruction; a machine instruction; a machine dependent instruction; a microcode; a firmware instruction; state-setting data; or either a source code or an object code written in any combination of one or more programming languages, including an object oriented programming language such as Smalltalk (registered trademark), JAVA (registered trademark), C++, or the like, and a conventional procedural programming language such as a “C” programming language or a similar programming language.

Computer-readable instructions may be provided to a processor of a general-purpose computer, special purpose computer, or other programmable data processing devices, or to programmable circuitry, locally or via a local area network (LAN), wide area network (WAN) such as the Internet, or the like, so that the computer-readable instructions are executed to create means for performing operations specified in the flowcharts or block diagrams. Examples of the processor include a computer processor, a processing unit, a microprocessor, a digital signal processor, a controller, a microcontroller, and the like.

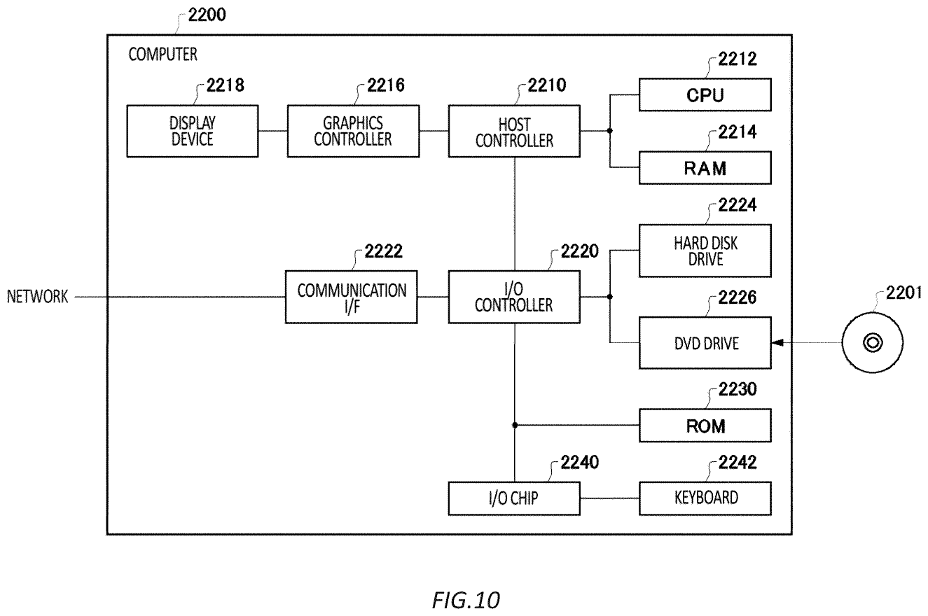

FIG. 10 shows an example of a computer 2200 in which a plurality of aspects of the present invention may be embodied in whole or in part. A program installed in the computer 2200 can cause the computer 2200 to function as an operation associated with the apparatuses according to the embodiments of the present invention or as one or more sections of the apparatuses, or can cause the operation or the one or more sections to be executed, and/or can cause the computer 2200 to execute a process according to the embodiments of the present invention or a stage of the process. Such programs may be executed by a CPU 2212 to cause the computer 2200 to perform specific operations associated with some or all of the blocks in the flowcharts and block diagrams described in the present specification.

The computer 2200 according to the present embodiment includes the CPU 2212, an RAM 2214, a graphics controller 2216, and a display device 2218, which are mutually connected by a host controller 2210. The computer 2200 also includes input/output units such as a communication interface 2222, a hard disk drive 2224, a DVD-ROM drive 2226, and an IC card drive, which are connected to the host controller 2210 via an input/output controller 2220. The computer also includes legacy input/output units such as an ROM 2230 and a keyboard 2242, which are connected to the input/output controller 2220 via an input/output chip 2240.

The CPU 2212 operates according to programs stored in the ROM 2230 and the RAM 2214, thereby controlling each unit. The graphics controller 2216 acquires image data generated by the CPU 2212 in a frame buffer or the like provided in the RAM 2214 or in itself, such that the image data is displayed on the display device 2218.

The communication interface 2222 communicates with other electronic devices via a network. The hard disk drive 2224 stores programs and data used by the CPU 2212 in the computer 2200. The DVD-ROM drive 2226 reads programs or data from a DVD-ROM 2201 and provides the programs or the data to the hard disk drive 2224 via the input/output controller 2220. The IC card drive reads the programs and the data from the IC card, and/or writes the programs and the data to the IC card.

The ROM 2230 stores therein boot programs and the like executed by the computer 2200 at the time of activation, and/or programs that depend on the hardware of the computer 2200. The input/output chip 2240 may also connect various input/output units to the input/output controller 2220 via a parallel port, a serial port, a keyboard port, a mouse port, or the like.

The program is provided by a computer-readable medium such as the DVD-ROM 2201 or the IC card. The program is read from a computer-readable medium, installed in the hard disk drive 2224, the RAM 2214, or the ROM 2230 which are also examples of the computer-readable medium, and executed by the CPU 2212. The information processing written in these programs is read by the computer 2200 and provides cooperation between the programs and the above-described various types of hardware resources. The apparatus or method may be constituted by implementing operations or processing of information according to use of the computer 2200.

For example, in a case where communication is performed between the computer 2200 and an external device, the CPU 2212 may execute a communication program loaded in the RAM 2214 and instruct the communication interface 2222 to perform communication processing based on a processing written in the communication program. Under the control of the CPU 2212, the communication interface 2222 reads transmission data stored in a transmission buffer processing area provided in a recording medium such as the RAM 2214, the hard disk drive 2224, the DVD-ROM 2201, or the IC card, transmits the read transmission data to the network, or writes reception data received from the network in a reception buffer processing area or the like provided on the recording medium.

In addition, the CPU 2212 may cause the RAM 2214 to read all or a necessary part of a file or database stored in an external recording medium such as the hard disk drive 2224, the DVD-ROM drive 2226 (DVD-ROM 2201), the IC card, or the like, and may execute various types of processing on data on the RAM 2214. Then, the CPU 2212 writes the processed data back in the external recording medium.

Various types of information such as various types of programs, data, tables, and databases may be stored in a recording medium and subjected to information processing.

The CPU 2212 may execute, on the data read from the RAM 2214, various types of processing including various types of operations, information processing, conditional judgement, conditional branching, unconditional branching, information retrieval/replacement, or the like described throughout the present disclosure and specified by instruction sequences of the programs, and writes the results back to the RAM 2214. In addition, the CPU 2212 may retrieve information in a file, a database, or the like in the recording medium. For example, when a plurality of entries, each having an attribute value of a first attribute associated with an attribute value of a second attribute, is stored in the recording medium, the CPU 2212 may retrieve, out of the plurality of entries, an entry with the attribute value of the first attribute specified that meets a condition, read the attribute value of the second attribute stored in said entry, and thereby acquiring the attribute value of the second attribute associated with the first attribute meeting a predetermined condition.

The programs or software modules described above may be stored in a computer-readable medium on the computer 2200 or near the computer 2200. In addition, a recording medium such as a hard disk or an RAM provided in a server system connected to a dedicated communication network or the Internet can be used as a computer-readable medium, thereby providing a program to the computer 2200 via the network.

While the present invention has been described by way of the embodiments, the technical scope of the present invention is not limited to the above-described embodiments. It is apparent to persons skilled in the art that various alterations or improvements can be made to the above-described embodiments. It is also apparent from the described scope of the claims that the embodiments to which such alterations or improvements are made can be included in the technical scope of the present invention.

It should be noted that the operations, procedures, steps, stages, or the like of each process performed by a device, system, program, and method shown in the claims, the specification, or the drawings can be performed in any order as long as the order is not indicated by “prior to,” “before,” or the like and as long as the output from a previous process is not used in a later process. Even if the process flow is described using phrases such as “first” or “next” in the claims, the specification, or the drawings for the sake of convenience, it does not necessarily mean that the process must be performed in this order.

### EXPLANATION OF REFERENCES

100 110 120 130: microfluidic device; 101: structure; 101a: obstacle; 201 to 203: observation light; 325: illumination driver; 326: motor driver; 300: observation apparatus; 310: observation optical system; 311: objective lens; 313: imaging lens; 314:

two-dimensional detector; 315: mask unit; 321: control unit; 322: memory; 323: input unit; 324: display unit; 330: first stage; 350 to 354: mask; 351a to 354a: transmissive region; 2200: computer; 2201: DVD-ROM; 2210: host controller; 2212: CPU; 2214: RAM; 2216: graphics controller; 2218: display device; 2220: input/output controller; 2222: communication interface; 2224: hard disk drive; 2226: DVD-ROM drive; 2230: ROM; 2240: input/output chip; 2242: keyboard.

What is claimed is:

1. An observation apparatus which observes a microfluidic device having an observation target region that is a target of observation, and a structure, the observation apparatus comprising:
an observation optical system and a control device, wherein
the observation optical system has
i) a mask which blocks at least a part of light that is subjected to an optical effect by the structure that exists partway on a path of a light beam reaching the observation optical system, in light beams from the observation target region, each of which is the light beam, the mask including a plurality of masks being masks in which regions that are blocked are different from each other, and being configured to be switchable, or
ii) a mask which blocks at least a part of light that is subjected to an optical effect by the structure that exists partway on a path of a light beam reaching the observation optical system, in light beams from the observation target region, each of which is the light beam, the mask being switchable in a state in which the regions that are blocked are different from each other, and
the control device automatically switches the mask according to a position of the observation target region or a type of the microfluidic device that is observed.

2. The observation apparatus according to claim 1, wherein
the observation optical system has an objective lens which collects a light beam from the observation target region, and
the mask is arranged at a position corresponding to a rear focal position of the objective lens.

3. The observation apparatus according to claim 1, further comprising: an illumination optical system which illuminates the observation target region.

4. The observation apparatus according to claim 1, wherein the mask is configured to be movable in a direction along an optical axis of the observation optical system.

5. The observation apparatus according to claim 1, wherein the mask which is switchable in a state in which the regions that are blocked are different from each other, is formed by a spatial light modulator.

6. The observation apparatus according to claim 1, wherein the mask is switched based on information received by the observation apparatus.
